(19) United States
(12) Patent Application Publication
(10) Pub. No.: US 2021/0213090 A1
(43) Pub. Date: Jul. 15, 2021

(54) COMPOSITIONS, USES AND METHODS FOR TREATMENT AND/OR PREVENTION OF ELEVATED CHOLESTEROL, HYPERCHOLESTEROLEMIA, AND CARDIOVASCULAR DISEASE

(71) Applicant: Nutraceutical Holdings Inc., St. Michael (BB)

(72) Inventors: Ravi Arora, Vancouver (CA); Amila Oshan Silva, Upland, CA (US)

(73) Assignee: Nutraceutical Holdings Inc., St. Michael (BB)

(21) Appl. No.: 17/153,496

(22) Filed: Jan. 20, 2021

Related U.S. Application Data
(63) Continuation of application No. PCT/IB2019/056126, filed on Jul. 17, 2019.
(60) Provisional application No. 62/701,035, filed on Jul. 20, 2018.
(60) Provisional application No. 62/784,152, filed on Dec. 21, 2018.

(51) Int. Cl. A61K 36/63; A61K 31/716; A61K 36/062; A61K 31/122; A61K 9/48; A61K 9/00; A61P 3/06; A23L 33/125; A23L 33/105; A23L 33/115; A23L 33/145; A23L 33/00

(52) U.S. Cl. CPC A61K 36/63; A61K 31/716; A61K 36/062; A61K 31/122; A61K 9/4808; A61K 9/0056; A23V 2002/00; A23L 33/125; A23L 33/105; A23L 33/115; A23L 33/145; A23L 33/40; A61P 3/06

(57) ABSTRACT

A composition for preventing and/or treating clinically elevated cholesterol levels, hypercholesterolemia and/or cardiovascular disease contains β-glucan, olive oil or olive leaf polyphenols or extra virgin olive oil (EVOO) or olive leaf extract (OLE), and red rice yeast substances (e.g. red rice yeast extract (RRYE)). Methods of using the composition to treat and/or prevent clinically elevated cholesterol levels, hypercholesterolemia and/or cardiovascular disease are provided.

### CROSS REFERENCE TO RELATED APPLICATIONS

This is a continuation of PCT application No. PCT/IB2019/056126, entitled “COMPOSITIONS, USES AND METHODS FOR TREATMENT AND/OR PREVENTION OF ELEVATED CHOLESTEROL, HYPERCHOLESTEROLEMIA, AND CARDIOVASCULAR DISEASE”, filed Jul. 17, 2019, which is incorporated herein by reference, which claims priority to, and the benefit of, U.S. provisional patent application Ser. Nos. 62/701,035, filed Jul. 20, 2018, and 62/784,152, filed Dec. 21, 2018, both of which are also incorporated herein by reference in their entireties for all purposes.

### BACKGROUND OF THE INVENTION

A characteristic pathogenic feature of hypercholesterolemia is the elevation of total cholesterol (TC) and low-density lipoprotein cholesterol (LDL-C) in the blood above 200 mg/dL and 100 mg/dL, respectively.¹ Approximately one third of global deaths are attributable to cardiovascular disease, and elevated blood cholesterol is among the most prevalent modifiable cardiovascular risk factors.² Despite this, the total annual cost of treating cardiovascular disease in the United States exceeds $300 billion, and hypercholesterolemia remains one of the major causes of morbidity and mortality amongst Americans.³ For example, the disease affects over 100 million American adults, of which less than 30% have their cholesterol levels under control.⁴

Current therapies for hypercholesterolemia include medical management, specifically with the use of a cholesterol-lowering drug class known as 3-hydroxy-3-methylglutaryl-CoA (HMG-CoA) reductase inhibitors, also known as statins.⁵ Statins block the formation of cholesterol in the liver and are recommended as the primary pharmacologic agent to achieve target blood cholesterol goals on the basis of morbidity and mortality outcome trials.⁶ Notwithstanding their success in reducing cholesterol levels, statin use poses significant challenges: their adverse effects and their cost.⁷ The most common adverse effects associated with statin use are myalgia and myopathy, reported in 10-29% of patients.⁸ Statin use is further associated with increased risk of diabetes and possible effects on cognition.⁹ Due to the high incidence of adverse events associated with statin drugs, up to 30% of patients eventually discontinue their statin pharmacotherapy.¹⁰ Meanwhile, the annual cost of statin treatment per patient ranges from $300 for generics to $1400 for non-generics, while the annual cost of other cholesterol lowering drug classes such as proprotein convertase subtilisin/kexin type 9 (PCSK9) inhibitors ranges up to $14,000 at wholesale price.¹¹ The addition of the latter drugs to the medicare formulary would increase expenditure by $125 billion dollars if all eligible patients are converted¹⁶.

There is longstanding evidence that nonpharmacologic interventions such as dietary management and supplementation can be cost-effective approaches to preventing cardiovascular disease.⁶ For example, clinical studies have reported that oats can lower LDL-C by about 5% over a 4-week period, while virgin olive oil and olive leaf extract (OLE) has been reported to lower LDL-C by about 3-6% over a 3-month period.¹² Meanwhile, supplementation with red rice yeast extract (RRYE) has also been reported in several clinical studies to reduce cholesterol by an average of 10% over a 6- to 24-month period.¹³ RRYE is obtained from the product of rice fermented with the red yeast, Monascus purpureus, and contains substances including monacolins and sterols which collectively inhibit cholesterol biosynthesis.¹⁴ However, none of these studies have shown the individual oatmeal, olive oil/OLE or RRYE interventions to adequately lower clinically elevated cholesterol levels to the target lipid goals set by the American Heart Association, nor is there evidence for the treatment of hypercholesterolemia with these dietary supplements alone at the total exclusion of other pharmacologic and non-pharmacologic interventions such as medical nutrition therapy, exercise, weight control, and smoking cessation.^{3,6,12-14} As a result, there remains a need for novel methods that can effectively and economically manage hypercholesterolemia and thus protect against the progression of cardiovascular disease, including non-pharmacologic approaches that may lower cholesterol.

The foregoing examples of the related art and limitations related thereto are intended to be illustrative and not exclusive. Other limitations of the related art will become apparent to those of skill in the art upon a reading of the specification and a study of the drawings.

### SUMMARY OF THE INVENTION

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

One aspect of the invention provides a composition for preventing and/or treating clinically elevated cholesterol levels, hypercholesterolemia and/or cardiovascular disease containing β-glucan, olive oil or olive leaf polyphenols or extra virgin olive oil (EVOO) or olive leaf extract (OLE), and red rice yeast substances.

In one aspect, the composition contains an effective amount of oat or barley β-glucan, extra virgin olive oil (EVOO) or olive leaf extract (OLE), and red rice yeast extract (RRYE). In some aspects, the composition further contains a pharmaceutically acceptable vehicle.

In some aspects, the weight percentage ratio of EVOO/OLE to oat or barley β-glucan to RRYE is about 0.75˜1.25 to about 1.25˜6.00 to about 0.75˜6.00.

In some aspects, the composition is provided in a dosage form comprising about 300 mg˜4.0 grams of oat or barley β-glucan, about 100 mg to 500 mg of OLE and about 400 mg˜4.0 grams of RRYE, or about 2.5˜3.5 grams of oat or barley β-glucan, about 3.0 to 5.0 mL of EVOO and about 500 mg˜3.6 grams of RRYE, or about 3.0 grams of oat or barley β-glucan, about 4.0 mL of EVOO and about 3.0 grams of RRYE.

In some aspects, the composition is provided in daily dosage forms comprising about 200 mg˜4.0 grams of oat or barley β-glucan, about 50 mg to 500 mg of OLE and about 400 mg˜4.0 grams of RRYE, or about 2.5˜3.5 grams of oat or barley β-glucan, about 3.0 to 5.0 mL of EVOO and about 500 mg˜3.6 grams of RRYE, or about 3.0 grams of oat or barley β-glucan, about 4.0 mL of EVOO and about 3.0 grams of RRYE. The daily dosage referred to above may be divided into multiple doses in the form of tablets, pills or other vehicle to be consumed by individuals with hypercholesterolemia.

In some aspects, the composition further comprises coenzyme Q10, for example about 90˜200 mg of coenzyme Q10.

In some aspects, the composition is administered to a mammalian subject by orally administering the composition to prevent and/or treat clinically elevated cholesterol levels, hypercholesterolemia and/or cardiovascular disease.

In some aspects, a daily dosage of the composition is at least about 800 mg˜1.2 g of RRYE, about 500 mg˜1.0 g of oat or barley β-glucan, and about 150 mg˜350 mg of OLE; or at least about 800 mg˜1.2 g of RRYE, about 500 mg˜1.0 g of oat or barley β-glucan, and about 3.0˜5.0 mL of EVOO.

In some aspects, the composition is provided in a food product form, and the food product is optionally a ready-to-eat cereal product, a cereal product mix, a ready-to-drink beverage, a beverage mix, a dairy product, a spreadable product or a gelatinous product.

In addition to the exemplary aspects and embodiments described above, further aspects and embodiments will become apparent by reference to the drawings and by study of the following detailed descriptions.

### BRIEF DESCRIPTION OF THE DRAWINGS

The above-mentioned and other features and advantages of this invention, and the manner of attaining them, will become more apparent and the invention will be better understood by reference to the following description of embodiments of the invention taken in conjunction with the accompanying drawings, wherein:

FIG. 1 shows a distribution of percent change histogram for reduction of LDL-C in all patients of the study group (n=68, mean percent change=−21.8%) following 6 months of treatment with the study regimen (daily oatmeal, EVOO and RRYE consumption) in comparison to the mean percent change of −24% reported following 6 months of treatment with pravastatin “Null Value”; and

FIG. 2 shows a distribution of percent change histogram for reduction of LDL-C in the highly compliant patients of the study group (n=36, mean percent change=−30.0%) following 6 months of treatment with the study regimen (daily oatmeal, EVOO and RRYE consumption).

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION OF THE INVENTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

Oatmeal contains a soluble dietary fiber, oat β-glucan, known in the art to lower cholesterol levels in the blood. 1.5 cups of cooked oatmeal contains approximately 3 grams of oat β-glucan. The chemical structure of oat β-glucan is as follows:

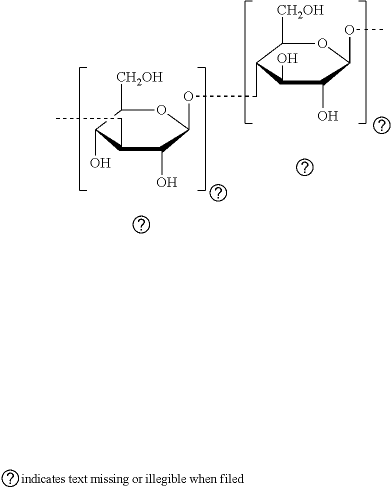

Barley is another source of β-glucan that has been shown to reduce cholesterol levels in the blood. Sources of β-glucan that have been scientifically accepted as reducing cholesterol levels in the blood include rolled oats (e.g. eaten as oatmeal), oat bran, whole oat flour, oatrim (hydrolyzed oat flour), whole grain barley and barley beta-fiber. In some embodiments, the oat or barley β-glucan is derived from whole oats or whole barley. In some embodiments, the oat or barley β-glucan is derived from processed oats or processed barley, e.g. rolled or crushed oats or barley. As used herein, the term “oat or barley β-glucan” includes the foregoing sources of β-glucan, as well as any source of β-glucan that is in future determined to have similar blood cholesterol lowering effects.

Extra virgin olive oil (EVOO)/olive leaf extract (OLE) contains olive oil polyphenols and derivatives thereof. EVOO and olive oil polyphenols are known in the art to reduce cholesterol levels in the blood and improve high-density lipoprotein functionality. As used herein, “olive oil or olive leaf polyphenols” mean compounds in olive oil and olive leaf extract with a polyphenolic substructure and antioxidant properties. Examples of olive oil or olive leaf polyphenols include phenolic alcohols (e.g. hydroxytyrosol and tyrosol), phenolic acids (e.g. vanillic, p-coumaric and ferulic acid), secoiridoids (e.g. oleuropein and ligstroside), lignans (e.g. pinoresinol), flavonoids (e.g. luteolin and apigenine), as well as derivatives and/or analogues of such compounds. In some embodiments, the olive oil or olive leaf polyphenols independently have one of the following chemical structures:

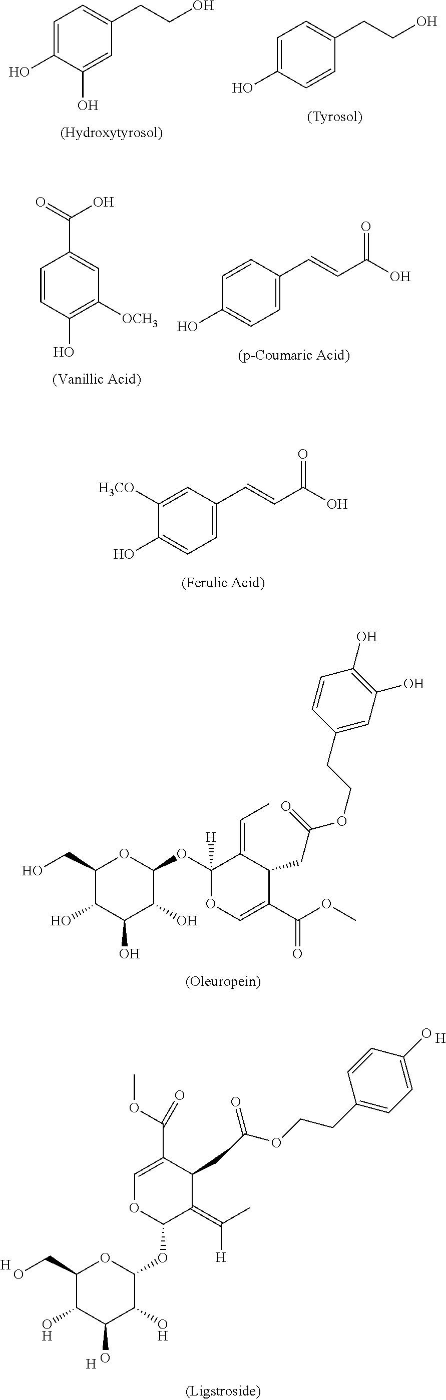

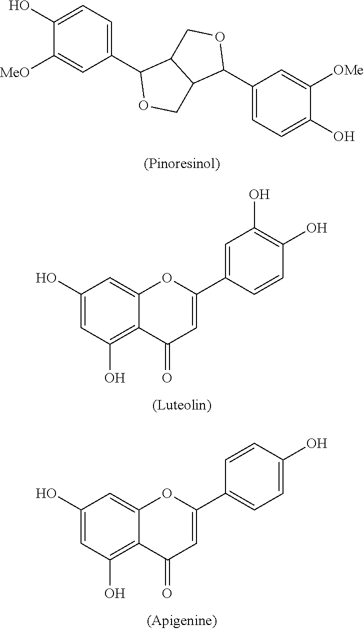 [SMILES: COc1cc(C2OCC3C(c4ccc(O)c(OC)c4)OCC23)ccc1O ; O=c1cc(-c2ccc(O)c(O)c2)oc2cc(O)cc(O)c12 ; O=c1cc(-c2ccc(O)cc2)oc2cc(O)cc(O)c12]

Red Rice Yeast Extract (RRYE) contains red yeast rice substances and derivatives thereof. RRYE and red rice yeast substances are known in the art to collectively inhibit cholesterol biosynthesis. As used herein, “red rice yeast substances” mean compounds found in red rice yeast with cholesterol-lowering properties. Examples of red rice yeast substances include monacolin compounds (e.g. monacolins K, J, L, M, X and their hydroxy-acid form; dehydromonacolin K, dihydromonacolin L, compactin, 3α-hydroxy-3,5-dihydromonacolin L and their hydroxy-acid form), sterols (e.g. β-sitosterol, campesterol, stigmasterol, and sapogenin), isoflavones and isoflavone glycosides, monounsaturated fatty acids (e.g. oleic acid), as well as derivatives and/or salts and/or analogues of such compounds. In some embodiments, the red rice yeast substances independently have one of the following chemical structures:

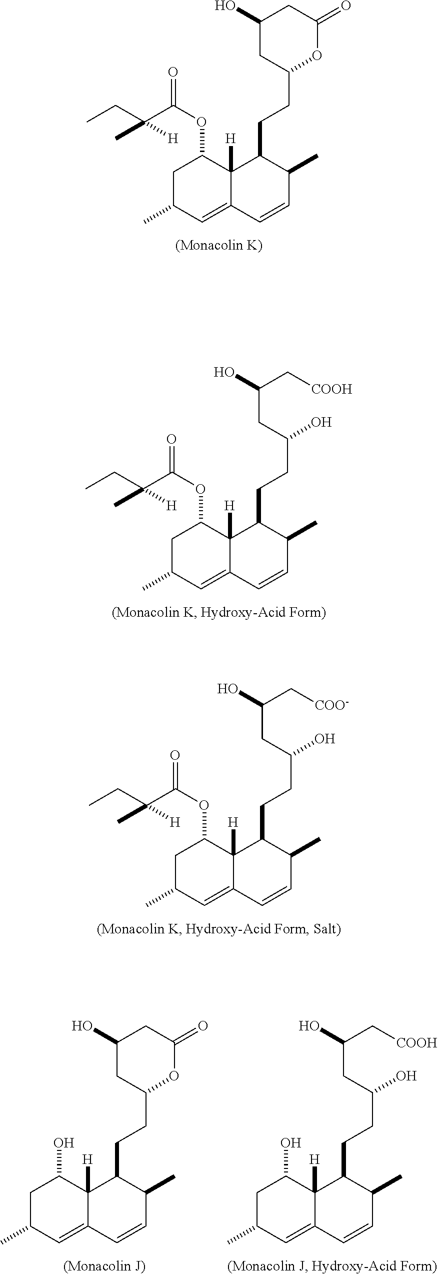

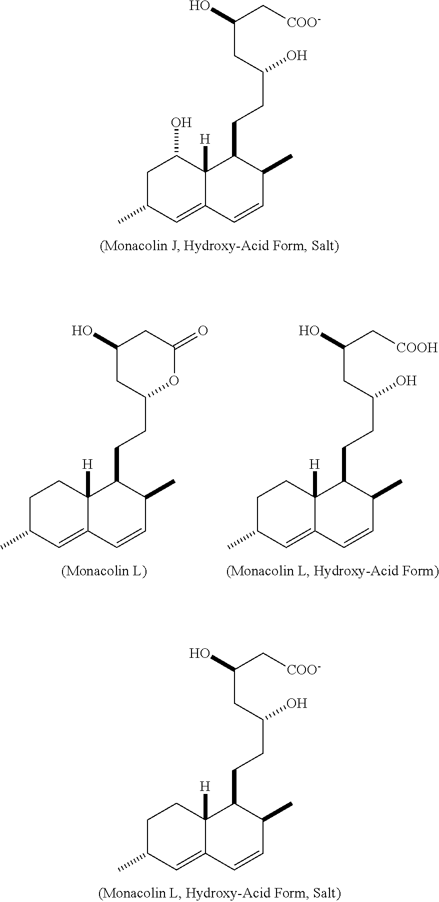

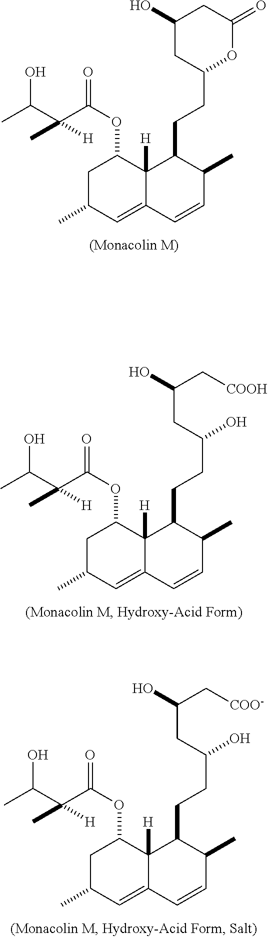

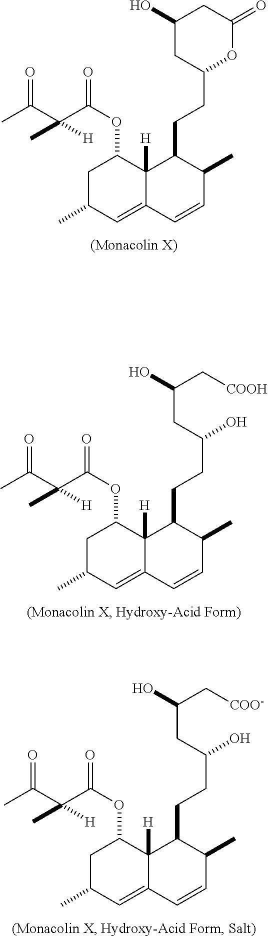

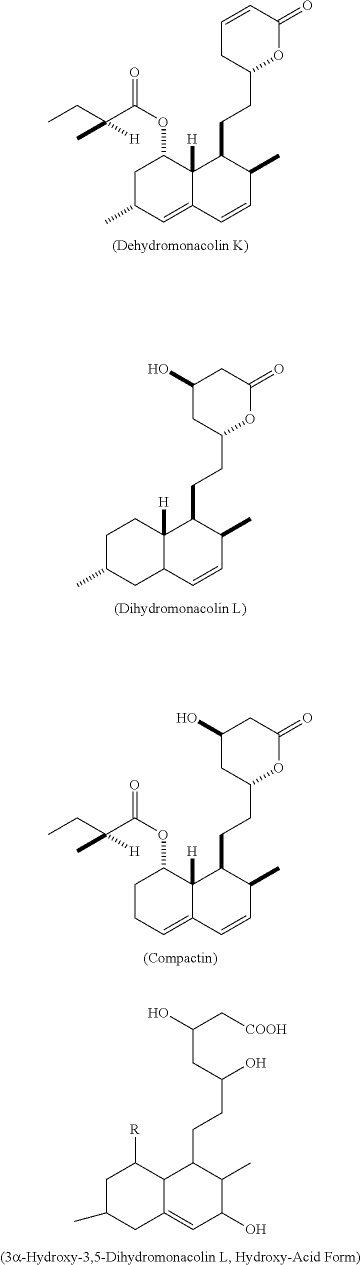

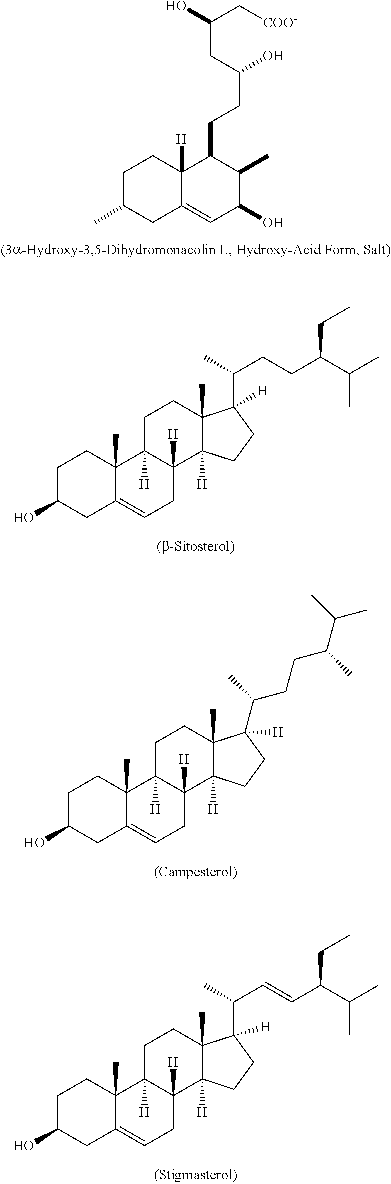

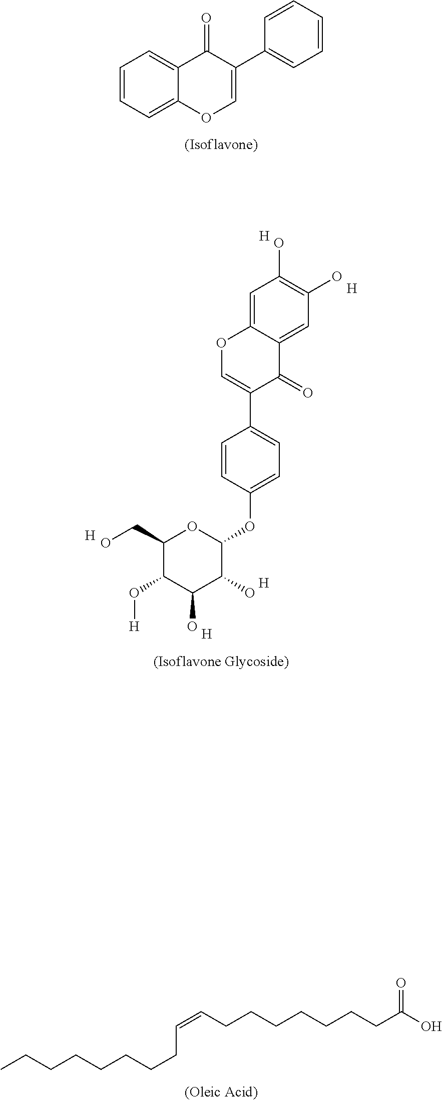

Coenzyme Q₁₀, also referred to as ubiquinone and having the following structure is a fat-soluble coenzyme that participates in aerobic cellular respiration. It has been shown that coenzyme Q₁₀ supplementation decreases statin-related mild-to-moderate muscle symptoms¹⁷.

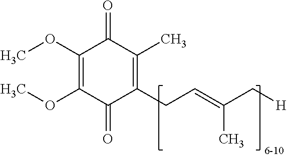

The inventors have found that treatment of patients suffering from clinically elevated cholesterol levels by concurrent administration of active agents from three naturally-derived dietary supplements, namely β-glucan (from oat or barley sources), olive oil or olive leaf polyphenols (from EVOO or OLE) and red rice yeast substances (from RRYE) produces a significant reduction in the lipid parameters of the disease. In particular, the inventors have unexpectedly found that these active agents, when administered concurrently to an individual suffering from clinically elevated cholesterol levels, produces a highly beneficial and synergistic clinical response via significant reduction of LDL-C and preservation of high-density lipoprotein cholesterol (HDL-C) compared to that obtained with administration of these active agents alone. In some embodiments, the reduction of LDL-C and preservation of HDL-C from concurrent administration of these three active agents was found to be superior to that obtained with a known prescription lipid-lowering medication administered alone. Based on the experimental results described herein, it can be soundly predicted that the concurrent administration of these three active agents will be useful in the prophylaxis and/or treatment of hypercholesterolemia and thus potentially lower the risk of cardiovascular disease.

The examples described herein demonstrate the unexpected effects of inhibiting or alleviating clinically elevated cholesterol levels by concurrently administering a therapeutically effective amount of oat or barley β-glucan, olive oil/olive leaf polyphenols and red rice yeast substances, each known individually in the art to be useful in modestly reducing cholesterol through mechanisms unique to each agent.

The utility of the present invention in the prophylaxis and/or treatment of clinically elevated cholesterol levels and hypercholesterolemia and/or cardiovascular disease is believed to be a novel approach. The current gold standard for treating hypercholesterolemia is administering a statin such as pravastatin, whose mean percent change for LDL-C of −24% following a 6-month treatment period was used as the null value comparison in the examples described herein¹⁵.

The term “effective amount” used herein refers to the amount of the combination of active agents sufficient to confer a desired prophylactic or therapeutic effect in a subject. In one aspect, an effective amount for inhibiting or alleviating clinically elevated cholesterol levels or hypercholesterolemia improves or reduces one or more symptoms, conditions or progression thereof. In some embodiments, the symptoms, conditions or progression are determined and evaluated using methods known in the art that measure various disease progress-related indexes, for example by analyzing blood cholesterol levels via fasting lipid profile (e.g. measured TC, triglycerides, and HDL-C; calculated LDL-C). In some embodiments, the effective amount is determined by persons skilled in the art evaluating, for example, the administration route and frequency, body weight and gender of the subject receiving the combination of active agents. In some embodiments, an effective amount of the combination of active agents is formulated with a pharmaceutically acceptable vehicle and administered to the subject.

The term “pharmaceutically acceptable” used herein means that the vehicle is known in the art as compatible with the combination of active agents while also being safe to the subject receiving the treatment. In some embodiments, the pharmaceutically acceptable vehicle is determined by persons skilled in the art evaluating, for example, the solubility of the combination of active agents, in said vehicle.

The terms “prevent”, “preventing”, “prevention” and “prophylactic” as used herein refers to arrest, delay of onset (i.e., the period prior to clinical manifestation of a disease) and/or reduction of the risk of developing or worsening in a subject a condition such as clinically elevated cholesterol levels, hypercholesterolemia and/or cardiovascular disease.

The terms “treat”, “treating”, “treatment” and “therapeutic” as used herein refers to an approach for obtaining desired clinical results. Desired clinical results can include, but are not limited to, reduction of clinically elevated cholesterol levels or alleviation of at least one symptom of hypercholesterolemia and/or cardiovascular disease. For example, treatment can be diminishment of at least one symptom of hypercholesterolemia and/or cardiovascular disease, diminishment of the extent of hypercholesterolemia and/or cardiovascular disease, stabilization of hypercholesterolemia and/or cardiovascular disease, delay or slowing of hypercholesterolemia and/or cardiovascular disease progression, diminishment of hypercholesterolemia and/or cardiovascular disease reoccurrence, prolonging survival with hypercholesterolemia and/or cardiovascular disease, or complete eradication of hypercholesterolemia and/or cardiovascular disease.

The term “subject” as used herein, refers to an individual to whom the combination of active agents is to be delivered (e.g. for preventative and/or treatment purposes). The term “subject” includes mammals, in particular humans.

Aspects of the invention relate to compositions comprising β-glucan, polyphenols and red rice yeast substances. In some embodiments the β-glucan may be oat or barley β-glucan. The source of oat β-glucan may be oatmeal, such as cooked oatmeal or derived from oats or barley, e.g. rolled oats, oat bran, whole oat flour, oatrim (hydrolyzed oat flour), whole grain barley, barley beta-fiber, or the like. In some embodiments the polyphenols may be olive oil polyphenols. The source of olive oil polyphenols may be EVOO or OLE. In some embodiments the source of red rice yeast substances may be RRYE. In particular embodiments the red rice yeast substances may be one or more of monacolin K or monacolin-related substances.

In some embodiments the composition of the present invention may comprise an effective amount of oat β-glucan, EVOO or OLE and RRYE. In some embodiments, the weight percentage ratio of oat or barley β-glucan to EVOO/OLE to RRYE may range from about 0.75˜1.25 to about 0.25˜6.00 to about 0.75˜6.00; about 0.75˜1.25 to about 0.25˜1.75 to about 0.75˜6.00; or about 0.85˜1.15 to about 1.35˜1.65 to about 0.85˜1.15; or about 0.95˜1.05 to about 1.40˜1.60 to about 0.95˜1.05; or about 1.0 to about 1.5 to about 1.0.

In some embodiments, the composition may be provided in an orally administrable form such as solid dosage forms (for example, capsules, tablets, pills and other such solid dosage forms known in the art) or liquid dosage forms (for example, emulsions, solutions, suspensions and other such liquid dosage forms known in the art). In other embodiments, the composition may be provided in an orally administrable form such as a combination of solid and liquid dosage forms (for example, a liquid-filled and semi-solid fill capsule within which one region comprises a liquid dosage form and another region comprises a solid dosage form, and other such combination liquid and solid dosage forms known in the art).

In some embodiments the composition may be provided as a food product, i.e. a readily edible or drinkable substance containing the combination of active agents of the present invention to provide a prophylactic and/or therapeutic effect. In particular embodiments the food product may be a ready-to-eat cereal product or a cereal product mix (e.g. muffins, cookies, breads, cakes, bars and the like), a ready-to-drink beverage or a beverage mix (e.g. smoothies, shakes, juices, a powder form and the like), a dairy product (e.g. yogurt, ice cream, frozen yogurt and the like), a spreadable product (e.g. spreadable fats, blended spreads and the like) a gelatinous product (e.g. gummies, gelatinous desserts and the like) or a chocolate product, for example a chocolate truffle product with a chocolate coating and a center filled with the composition that reduces cholesterol.

In embodiments where the present invention is provided as a food product, the food product may optionally include one or more of flavoring agents, sweeteners, texturizing agents, vitamins, minerals and other conventional additives. Flavoring agents may include cocoa, vanilla, fruit flavours and the like. Sweeteners may include natural sweeteners, such as, sucrose (e.g., cane sugar or sugar beet), honey, high fructose corn syrup, molasses, maple syrup, brown rice syrup, fruit juice sweeteners, barley malt, Stevia and the like, as well as artificial sweeteners, such as, sucralose, aspartame, saccharin and the like. Texturizing agents may include starch, modified starch (e.g., gelatinized starch), gum arabic, alginate, carboxymethyl cellulose, gelatin, guar gum, locust bean gum, pectin, and yellow gum. Vitamins may include vitamin A, vitamin B1, vitamin B2, pantothenic acid, vitamin B6, biotin, folic acid, vitamin B12, vitamin C, vitamin D, vitamin E, and vitamin K. Minerals may include calcium, iodine, iron, magnesium, manganese, phosphorus, potassium, selenium, sodium and zinc.

The composition may be provided in unit dosage form sufficient to be taken once a day or multiple times per day (e.g. twice or thrice a day). In some embodiments, total daily dosage may range from about 300 mg˜4.0 grams of oat or barley β-glucan, about 100 mg˜1.0 grams of OLE, and about 0.4˜4.0 grams of RRYE; about 1.0˜4.0 grams of oat or barley β-glucan, about 2.0 to 6.0 mL of EVOO (or between about 500 mg to about 1000 mg of OLE) and about 0.5˜4.0 grams of RRYE, or about 1.0˜3.5 grams of oat or barley β-glucan, about 3.0 to 5.0 mL of EVOO and about 2.4˜3.6 grams of RRYE, or about 3.0 grams of oat or barley β-glucan, about 4.0 mL of EVOO and about 3.0 grams of RRYE. Other embodiments of dosage forms might contain 1.0˜4.0 grams of oat or barley β-glucan, about 100 mg to 1000 mg of OLE and about 1.0˜4.0 grams of RRYE. In embodiments where the composition is provided as a food product, oat or barley β-glucan may be provided in the form of oatmeal, such as uncooked or cooked oatmeal with possible addition of oat or barley β-glucan in its powdered form such that the total amount of β-glucan is between 1.0-4.0 grams per unit dosage.

All of the dosage ranges stated herein include any intervening value and/or intervening sub-range and adjacent values that achieve substantially similar effects, including without limitation a unit dosage form containing 200 mg, 300 mg, 400 mg, 500 mg, 600 mg, 700 mg, 800 mg, 900 mg, 1.0 g, 1.1 g, 1.2 g, 1.3 g, 1.4 g, 1.5 g, 2.0 g, 2.5 g, 3.0 g or 3.5 g grams of oat or barley β-glucan; 2.5, 3.0, 3.5, 4.0, 4.5, 5.0 or 5.5 mL of EVOO and/or 50, 100, 150, 200, 300, 400, 500, 600, 700, 800 or 900 mg of OLE or some equivalent combination of EVOO and OLE; and 0.5, 0.6, 0.7, 0.8, 0.9, 1.0, 1.5, 2.0, 2.5, 3.0, or 3.5 grams of RRYE.

Aspects of the invention relate to methods of treating and/or preventing clinically elevated cholesterol levels and hypercholesterolemia and/or cardiovascular disease, comprising concurrently orally administering to a subject in need thereof β-glucan, polyphenols and red rice yeast substances. In some embodiments the β-glucan may be oat or barley β-glucan. The source of oat or barley β-glucan may be oats, for example in the form of whole oat groats, cut oats (e.g. steel cut oats or Scottish oatmeal), rolled oats (e.g. regular or instant), barley or oat flour, oatrim (hydrolyzed oat flour), whole grain barley, or barley beta-fiber. Cut oats and rolled oats may be cooked, to provide cooked oatmeal as a source of oat β-glucan. In some embodiments the polyphenols and may be olive oil or olive leaf polyphenols. The source of olive oil or olive leaf polyphenols may be olive oils, such as EVOO, virgin olive oil, refined olive oil, pomace oil or OLE. In some embodiments the source of red rice yeast substances may be RRYE. In particular embodiments the red rice yeast substances may be one or more of monacolin K or monacolin-related substances.

In some embodiments, the method of the present invention may comprise concurrently orally administering oat β-glucan, EVOO/OLE and RRYE to subjects in need thereof. The subject may have elevated cholesterol levels and hypercholesterolemia and/or cardiovascular disease. The weight percentage ratio of oat or barley β-glucan to EVOO/OLE to RRYE administered may range from about 0.75˜1.25 to about 0.25˜1.75 to about 0.75˜6.00, or about 0.85˜1.15 to about 1.35˜1.65 to about 0.85˜1.15, or about 0.95˜1.05 to about 1.40˜1.60 to about 0.95˜1.05, or about 1.0 to about 1.5 to about 1.0. Oral administration may comprise administering a composition as described herein. In some embodiments, total daily dosage may range from about 1.0˜4.0 grams of oat or barley β-glucan, about 2.0 to 6.0 mL of EVOO (or 500 mg to 1000 mg of OLE) and about 0.5˜4.0 grams of RRYE, or about 2.5˜3.5 grams of oat or barley β-glucan, about 3.0 to 5.0 mL of EVOO (or 500 mg to 1000 mg of OLE) and about 2.4˜3.6 grams of RRYE, or about 3.0 grams of oat or barley β-glucan, about 4.0 mL of EVOO (or 500 mg to 1000 mg of OLE) and about 3.0 grams of RRYE.

All of the dosage ranges stated herein include any intervening value and/or intervening sub-range or adjacent value that achieves substantially similar effects, including without limitation a daily dosage of 200 mg, 300 mg, 400 mg, 500 mg, 600 mg, 700 mg, 800 mg, 900 mg, 1.0 g, 1.1 g, 1.2 g, 1.3 g, 1.4 g, 1.5 g, 2.0 g, 2.5 g, 3.0 g or 3.5 g of oat or barley β-glucan per day; 2.5, 3.0, 3.5, 4.0, 4.5, 5.0 or 5.5 mL of EVOO and/or 50, 100, 150, 200, 300, 400, 500, 600, 700, 800 or 900 mg of OLE or some equivalent combination of EVOO and OLE per day; and 0.5, 0.6, 0.7, 0.8, 0.9, 1.0, 1.5, 2.0, 2.5, 3.0, or 3.5 grams of RRYE per day.

In one example embodiment, the composition is provided in a dosage form having 300 mg˜4.0 grams of oat or barley β-glucan, about 100 mg to 500 mg of OLE and about 500 mg˜4.0 grams of RRYE, or about 2.5˜3.5 grams of oat or barley β-glucan, about 3.0 to 5.0 mL of EVOO and about 500 mg˜3.6 grams of RRYE, or about 3.0 grams of oat or barley β-glucan, about 4.0 mL of EVOO and about 3.0 grams of RRYE. In some embodiments, a method of treating or preventing clinically elevated cholesterol levels, hypercholesterolemia and/or cardiovascular disease includes administering such a dosage form once daily or twice daily to a mammalian subject to achieve a desired daily dosage of at least about 300 mg˜4.0 grams of oat or barley β-glucan, about 100 mg to 500 mg of OLE and about 500 mg˜4.0 grams of RRYE.

All of the dosage ranges stated herein include any intervening value and/or intervening sub-range, including without limitation a dosage form containing or a daily dosage of 200 mg, 300 mg, 400 mg, 500 mg, 600 mg, 700 mg, 800 mg, 900 mg, 1.0 g, 1.1 g, 1.2 g, 1.3 g, 1.4 g, 1.5 g, 2.0 g, 2.5 g, 3.0 g, or 3.5 g of oat or barley β-glucan; about 50, 100, 150, 200, 300, 400, 500, 600, 700, 800, 900 or 1000 mg of OLE or about 2.5, 3.0, 3.5, 4.0, 4.5, 5.0 or 5.5 mL of EVOO or some equivalent combination of both OLE and EVOO; and about 400 mg, 500 mg, 600 mg, 700 mg, 800 mg, 900 mg, 1.0 g, 1.5 g, 2.0 g, 2.5 g, 3.0 g, or 3.5 g of RRYE.

In one example embodiment, a dosage form according to any embodiment described herein is administered orally to a mammalian subject, which may be a human, to treat or prevent clinically elevated cholesterol levels, hypercholesterolemia and/or cardiovascular disease. In some example embodiments, a dosage form according to any embodiment described herein is administered orally to a mammalian subject twice daily.

In some embodiments, the composition includes coenzyme Q10. In some embodiments, the composition is provided in a dosage form containing between 90˜200 mg of coenzyme Q10.

Embodiments of the present invention are further described with reference to the following examples, which are intended to be illustrative and not limiting in nature.

### Example 1

Male and female patients aged 40-70 years with TC ranging from 200-270 mg/dL and LDL-C ranging from 115-200 mg/dL were enrolled in this non-randomized interventional prospective study. Prior to study enrollment, all patients underwent assessment for eligibility via performance of a screening fasting lipid profile. The following patients were excluded from study enrollment: 1) those who were treated previously for hypercholesterolemia and had abnormal liver function test results; 2) those with a history of statin-associated myositis or rhabdomyolysis, myocardial infarction, stent, or coronary artery bypass grafting; 3) those taking any lipid-regulating drugs, hormone replacement therapy, drugs known to affect lipid concentrations, or supplements known to affect lipid levels; 4) those with an endocrine disease known to lead to lipid abnormalities; and 5) those who self-reported pregnancy, lactation, current smoking, prevalent heart disease, or cancer.

After enrollment, conditions for withdrawal were as follows: 1) those who wished to withdraw at any time for any reason; 2) those who developed an adverse reaction to any of the dietary supplements of the present invention; and 3) those who developed any serious medical conditions (e.g. heart disease and cancer) during the course of the study.

90 patients were enrolled in the study and 74 of these patients completed 3 months of the 6-month study protocol. Of these 74 patients, 68 patients (hereinafter known as “study patients”) completed the entire 6-month study protocol.

All study patients underwent a 6-month study protocol consisting of concurrent oral treatment with three distinct dietary supplements. Study patients were instructed to consume each of the three dietary supplements as an oral treatment for 6 months but were not otherwise instructed to alter their diet. The dietary supplements were: 1) 1.5 cups of cooked oatmeal once a day; 2) 2400 mg (if study patient TC<230 mg/dL) or 3600 mg (if study patient TC>230 mg/dL) of RRYE once a day and 3) Exclusive use of EVOO while cooking (maximum 30 mL/day). Study patients were also instructed to maintain a food diary tracking their daily intake of each of the three dietary supplements.

Fasting lipid profiles of all study patients were measured at baseline, 3 months, and 6 months to monitor the response to the study protocol.

Compliance of all study patients was assessed and stratified using telephone interviews and the food diary. Following the study's end, all study patients were divided into two groups based on patient compliance: 1) those who were highly compliant; and 2) those who were not. Study patients categorized as highly compliant were those who were identified as being complaint with the study protocol on at least 6 out of 7 days every week. The demographic information of the study patients is shown in Table 1.

TABLE 1
Demographic Information
Study Patients

| | |
|---|---|
| All | 68 |
| Highly Compliant | 36 |

Gender

| | |
|---|---|
| Female (%) | 40 (58.8%) |
| Male (%) | 28 (41.2%) |

Age

| | |
|---|---|
| Range | 40-70 |
| Mean (SD) | 56.6 (10.06) |

Ethnicity

| | |
|---|---|
| White | 18 (26.5%) |
| Black | 8 (11.8%) |
| Hispanic | 29 (42.6%) |
| Asian/Pacific Islander | 9 (13.2%) |
| Others/Unknown | 4 (5.9%) |

The daily intake of each of the three dietary supplements (oatmeal, RRYE and EVOO) was recorded in a food diary by each study patient as previously described. The study patients' average daily consumption of EVOO was 4 mL/day and was calculated from the food diaries.

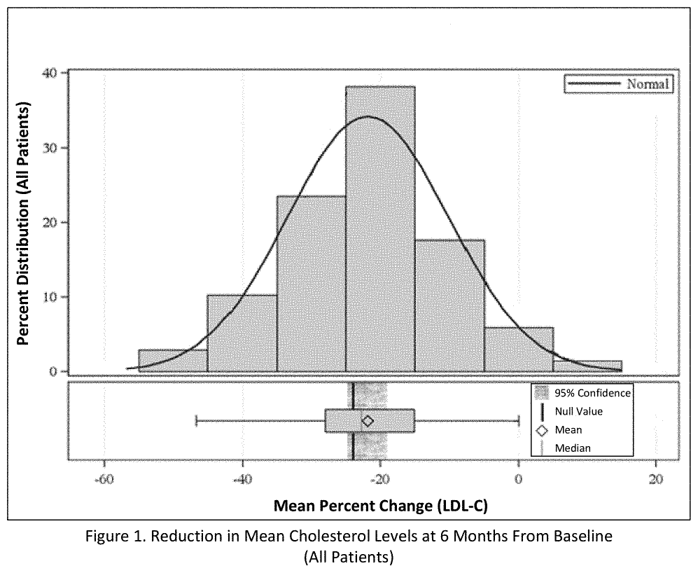

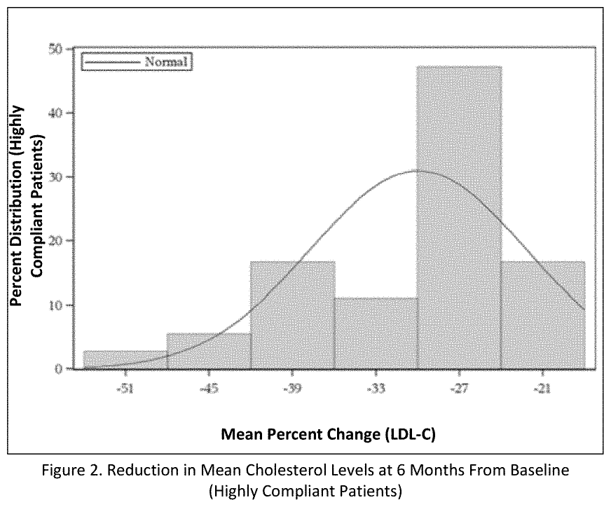

Results of the evaluation of percent change in mean cholesterol levels at 6 months from baseline are shown in FIGS. 1 and 2 and Table 2, with mean results summarized in Tables 3 and 4. Fasting cholesterol levels were measured at baseline, 3 months, and 6 months as previously described. For each cholesterol variable (e.g. TC, LDL-C, HDL-C, and triglycerides), the percent change in mean cholesterol level at 6 months for each study patient was calculated from baseline and then averaged.

A substantial reduction in LDL-C and TC was observed while HDL-C was preserved after 6 months of the study regimen for all study patients. 90% of females and 80% of males demonstrated a clinically significant reduction of TC and LDL-C. The reduction in LDL-C was 21.8% for all study patients and 30.0% for highly compliant patients, while the reduction in TC was 15.8% for all study patients and 21% for highly compliant patients. Prior studies have shown statins to reduce LDL-C by at least 15%, while a 6 month course of pravastatin therapy has been shown to reduce LDL-C by 24%.^{6,15} Using pravastatin as the null value (mean percent change=−24%) against which the study protocol for all study patients (−21.8%) was analyzed, there was no statistically significant difference between pravastatin and the study protocol on the percent reduction of LDL-C. However, when comparing pravastatin to the highly compliant patients (mean percent change=−30%), there was a statistically significant difference between pravastatin's beneficial effects on LDL-C and the study protocol. This demonstrates that the reduction in LDL-C after 6 months of the study regimen is at least statistically similar, and at best significantly better, to that of pravastatin, a current gold standard therapy for hypercholesterolemia.

TABLE 2
Percent Change in Mean Cholesterol Levels at 6 Months
from Baseline Following Study Protocol

| | | | |
|---|---|---|---|
| Variable | N-Size | Mean | SD |

All Study Patients

| | | | |
|---|---|---|---|
| TC | 68 | −15.8 | 8.2 |
| LDL-C | 68 | −21.9 | 11.7 |
| HDL-C | 68 | −1.8 | 9.8 |
| Triglycerides | 67 | −0.2 | 36.4 |

Highly Compliant Patients

| | | | |
|---|---|---|---|
| TC | 68 | −21.4 | 5.1 |
| LDL-C | 68 | −30.0 | 7.7 |
| HDL-C | 68 | −1.4 | 8.4 |
| Triglycerides | 67 | −12.1 | 27.2 |

TABLE 3
Change in Mean Cholesterol Levels at 6 Months from
Baseline Following Study Protocol.

| | | | |
|---|---|---|---|
| Variable | N-Size | Mean | SD |

All Study Patients

| | | | |
|---|---|---|---|
| TC | 68 | −36.5 | 19.7 |
| LDL-C | 68 | −33 | 18.1 |
| HDL-C | 68 | −1.5 | 5.4 |
| Triglycerides | 67 | −12.4 | 48.4 |

Highly Compliant Patients

| | | | |
|---|---|---|---|
| TC | 68 | −49.5 | 13.0 |
| LDL-C | 68 | −45.3 | 12.2 |
| HDL-C | 68 | −1.2 | 4.7 |
| Triglycerides | 67 | −27.6 | 48 |

TABLE 4
Detailed Results for Mean Cholesterol Level Change at
6 Months from Baseline Following Study Protocol.

| | | | |
|---|---|---|---|
| Variable | N-Size | Mean | SD |

All Study Patients

| | | | |
|---|---|---|---|
| TC | 68 | 232.9 | 18.13 |
| LDL-C | 68 | 152.3 | 16.62 |
| HDL-C | 68 | 55.4 | 15.27 |

Highly Compliant Patients

| | | | |
|---|---|---|---|
| TC | 68 | 196.4 | 19.69 |
| LDL-C | 68 | 119.3 | 18.12 |
| HDL-C | 68 | 55.4 | No Significant |
| | | | Change |

### Conclusion Based on Example 1

Briefly, the study regimen showed unexpected improvement in cholesterol levels. Taken alone, the study regimen's three active agents are previously known to reduce LDL-C at modest levels (e.g. Oatmeal at 5%, EVOO at 3˜5%, and RRYE at 10%).¹²⁻¹³ Based on an additive effect, the expected LDL-C reduction when taking the three active agents concurrently would be about 20%. However, the actual LDL-C reduction is about 30% in highly compliant patients—a highly beneficial and synergistic clinical response.

The study regimen resulted in a reduction or mean percent change in TC and LDL-C at 6 months from baseline for all study patients that was at least statistically similar or at best significantly better than that of a current, first-line gold standard therapy for hypercholesterolemia. Meanwhile, for highly compliant patients, the study regimen resulted in a statistically significant reduction in TC and LDL-C at 6 months from baseline in comparison to pravastatin. The HDL-C was preserved for all study patients. This means that some embodiments of the present invention have potential utility as a natural product replacement for existing treatments, without the risk of side effects posed by current clinical treatments.

### Example 2

Based on the above study results, a novel supplement was manufactured with all three components of the study protocol combined into a single pill. The pill contained 500 mg of RRYE, 375 mg of oat beta glucan and 125 mg of olive leaf extract. A study was done where patients with hypercholesterolemia were asked to consume the pills twice a day. Patients experienced no side-effects from consumption of the pills. The goal of the study was to assess whether the combination of the three ingredients could lower LDL-C levels by at least 15%, which is clinically significant response. Fasting LDL-C were obtained prior to study enrollment and after 4 weeks of enrollment in the study. Preliminary results show that after consumption of the pills for only 4 weeks, patients reduced their LDL-C by an average 20%, which is superior to the effect achieved by low doses of the current first line clinical therapy.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are consistent with the broadest interpretation of the specification as a whole.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

### REFERENCES

The following references are of interest with respect to the subject matter described herein. Each of the following references is incorporated in its entirety herein for all purposes.
1. Expert Panel on Detection, Evaluation, and Treatment of High Blood Cholesterol in Adults, Executive Summary of the Third Report of the National Cholesterol Education Program (NCEP) Expert Panel on Detection, Evaluation, and Treatment of High Blood Cholesterol in Adults (Adult Treatment Panel III), JAMA, 285(19):2486-97 (2001).
2. GBD 2013 Mortality and Causes of Death Collaborators, Global, regional, and national age-sex specific all-cause and cause-specific mortality for 240 causes of death, 1990-2013: a systematic analysis for the Global Burden of Disease Study 2013, Lancet, 385(9963):117-71 (2015); Nilsson P M et al, Population-attributable risk of coronary heart disease risk factors during long-term follow-up: the Malmö Preventive Project, J Intern Med, 260(2):131-41(2006).
3. Benjamin E J et al, Heart Disease and Stroke Statistics—2018 Update: A Report from the American Heart Association, Circulation, 137:67-492 (2018); Nicholls S and Lundman P, The emerging role of lipoproteins in atherogenesis: beyond LDL cholesterol, Semin Vasc Med, 4:187-95 (2004).
4. Mozaffarian D et al, Heart Disease and Stroke Statistics—2016 Update: A Report from the American Heart Association, Circulation, 133:e38-e360 (2016).
5. Gotto A M Jr, Statins: Power Drugs for Lowering Cholesterol, Circulation, 105:1514-16 (2002).
6. Jellinger P S et al, American Association of Clinical Endocrinologists and American College of Endocrinology Guidelines for Management of Dyslipidemia and Prevention of Cardiovascular Disease (AACE 2017 Guidelines), Endocr Pract, 23(4):479-97 (2017).
7. Kashani A et al, Risks associated with statin therapy: a systematic overview of randomized clinical trials, Circulation, 114:2788-97 (2006).
8. Jacobson T A et al, Provider recommendations for patient-reported muscle symptoms on statin therapy: Insights from the Understanding Statin Use in America and Gaps in Patient Education survey, J Clin Lipidol, 12(1):78-88 (2018).
9. Sattar N et al, Statins and risk of incident diabetes: a collaborative meta-analysis of randomised statin trials, Lancet, 375:735-42 (2010); Mailman T et al, Inhibition of neuronal cholesterol biosynthesis with lovastatin leads to impaired synaptic vesicle release even in the presence of lipoproteins or geranylgeraniol, J Neurochem, 119:1002-15 (2011).
10. Downs et al, Primary prevention of acute coronary events with lovastatin in men and women with average cholesterol levels: Results of AFCAPS/TexCAPS, JAMA, 279:1615-22 (1998); Rosenthal R L, Effectiveness of altering serum cholesterol levels without drugs, Proc (Bayl Univ Med Cent), 13(4):351-55 (2000).
11. Jick H et al, Comparison of prescription drug costs in the United States and the United Kingdom, Part 1: Statins, Pharmacotherapy, 32(1):1-6 (2012); Shravanthi R G et al, Cost-Effectiveness of LDL-C Lowering with Evolocumab in Patients with High Cardiovascular Risk in the United States, Clin Cardiol, 39(6):313-20 (2016).
12. Wolever T et al, Physicochemical properties of oat β-glucan influence its ability to reduce serum LDL cholesterol in humans: a randomized clinical trial, Am J Clin Nutr, 92(4):723-32 (2010); Gulati S et al, Effects of 3 g of soluble fiber from oats on lipid levels of Asian Indians—A randomized controlled, parallel arm study, Lipids Health Dis, 16(1):71 (2017); Estruch R et al, Effects of a Mediterranean style diet on cardiovascular risk factors: A randomized trial, Ann Intern Med, 145(1):1-11 (2006). Lockyer S et al. Impact of phenolic-rich olive leaf extract on blood pressure, plasma lipids and inflammatory markers: a randomised controlled trial. European Journal of Nutrition, 56(4): 1421-1432 (2017).
13. Lu Z et al, Effect of Xuezhikang, an extract from red yeast Chinese rice, on coronary events in a Chinese population with previous myocardial infarction, Am J Cardiol, 101:1689-93 (2008); Li Y et al, A meta-analysis of red yeast rice: An effective and relatively safe alternative approach for dyslipidemia, PLoS One, 9(6):e98611 (2014).
14. Heber D et al, Cholesterol-lowering effects of a proprietary Chinese red-yeast-rice dietary supplement, Am J Clin Nutr, 69(2):231-6 (1999); Li Y G et al, Identification and chemical profiling of monacolins in red yeast rice using high-performance liquid chromatography with photodiode array detector and mass spectrometry, J Pharm Biomed Anal, 35(5):1101-12(2004).
15. Wolffenbuttel B H et al, Efficacy and safety of a new cholesterol synthesis inhibitor, atorvastatin, in comparison with simvastatin and pravastatin, in subjects with hypercholesterolemia, Neth J Med, 52(4):131-7 (1998).
16. Kazi D et al, Cost-effectiveness of PCSK9 Inhibitor Therapy in Patients With Heterozygous Familial Hypercholesterolemia or Atherosclerotic Cardiovascular Disease. JAMA. 2016; 316(7):743-753.
17. Ajda Skarlovnik et al Coenzyme Q10 Supplementation Decreases Statin-Related Mild-to-Moderate Muscle Symptoms: A Randomized Clinical Study. Med Sci Monit. 2014; 20: 2183-2188.

What is claimed is:

1. A composition for preventing and/or treating clinically elevated cholesterol levels, hypercholesterolemia and/or cardiovascular disease, the composition comprising:
an effective amount of β-glucan, olive oil or olive leaf polyphenols, and red rice yeast substances.

2. The composition according to claim 1, wherein the β-glucan comprises oat or barley β-glucan, the olive oil or olive leaf polyphenols comprises extra virgin olive oil (EVOO) or olive leaf extract (OLE), the red rice yeast substances comprise red rice yeast extract (RRYE), and further comprises a pharmaceutically acceptable vehicle.

3. The composition according to claim 2, wherein the weight percentage ratio of EVOO/OLE to oat or barley β-glucan to RRYE is about 0.75˜1.25 to about 1.25˜6.00 to about 0.75˜6.00.

4. The composition according to claim 3, wherein the composition is provided in a dosage form comprising about 200 mg˜4.0 grams of oat or barley β-glucan, about 50 mg to 500 mg of OLE and about 400 mg˜4.0 grams of RRYE.

5. The composition according to claim 4, wherein the composition is provided in a dosage form comprising about 300˜500 mg of oat or barley β-glucan, about 100˜200 mg of OLE, and about 400˜600 mg of RRYE.

6. The composition according to claim 3, wherein the composition is provided in a dosage form comprising about 2.5˜3.5 grams of oat or barley β-glucan, about 3.0 to 5.0 mL of EVOO and about 500 mg˜3.6 grams of RRYE.

7. A composition according to claim 6, wherein the composition is provided in a dosage form comprising about 3.0 grams of oat or barley β-glucan, about 4.0 mL of EVOO and about 3.0 grams of RRYE.

8. The composition according to claim 1, further comprising coenzyme Q10.

9. The composition according to claim 8, wherein the composition is provided in a dosage form comprising about 90˜200 mg of coenzyme Q10.

10. The composition according to claim 1, wherein the composition is provided in a solid dosage form selected from the group consisting of capsules, tablets and pills, or in a liquid dosage form selected from the group consisting of emulsions, solutions and suspensions.

11. The composition according to claim 1, wherein the composition is provided in a combination of solid and liquid dosage forms contained within a single capsule.

12. The composition according to claim 11, wherein the single capsule is a liquid-filled and semi-solid fill capsule within which one region comprises a liquid dosage form and another region comprises a solid dosage form.

13. The composition according to claim 1, wherein the composition is provided in a food product form.

14. The composition according to claim 13, wherein the food product form is selected from the group consisting of a ready-to-eat cereal product, a cereal product mix, a ready-to-drink beverage, a beverage mix, a dairy product, a spreadable product, a gelatinous product, or a chocolate product.

15. The composition according to claim 13, wherein the food product form is a ready-to-eat form or a pre-mix form.

16. The composition according to claim 2, wherein the source of the oat or barley β-glucan comprises whole oats, whole barley, processed oats, processed barley, oat bran, oat flour, oatrim, barley flour, barley beta-fiber or oatmeal.

17. A method of preventing and/or treating clinically elevated cholesterol levels, hypercholesterolemia and/or cardiovascular disease in a subject comprising orally administering to the subject a composition as defined in claim 2.

18. A method of preventing and/or treating clinically elevated cholesterol levels, hypercholesterolemia and/or cardiovascular disease in a subject comprising orally administering to the subject a unit dosage form comprising a composition as defined in claim 2 twice daily.

19. A method of preventing and/or treating clinically elevated cholesterol levels, hypercholesterolemia and/or cardiovascular disease in a subject as defined in claim 17 comprising administering to the subject a daily dosage of about 800 mg˜1.2 g of RRYE, about 500 mg˜1.0 g of oat or barley β-glucan, and about 150 mg˜350 mg of OLE, or a daily dosage of about 400 mg˜1.2 g of RRYE, about 300 mg˜1.0 g of oat or barley β-glucan, and about 100 mg˜350 mg of OLE, or a daily dosage of about 800 mg˜1.2 g of RRYE, about 500 mg˜1.0 g of oat or barley β-glucan, and about 3.0˜5.0 mL of EVOO.
